(19) United States
(12) Patent Application Publication
(10) Pub. No.: US 2009/0048346 A1
(43) Pub. Date: Feb. 19, 2009

(54) THERAPEUTIC FORMULATIONS

(75) Inventors: Gaylen M. Zentner, Salt Lake City, UT (US); Chung Shih, Sandy, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/721,662

(22) PCT Filed: Nov. 30, 2005
(86) PCT No.: PCT/US2005/043481

§ 371 Date: Jun. 27, 2008

Related U.S. Application Data
(60) Provisional application No. 60/632,335, filed on Nov. 30, 2004.

(51) Int. Cl. A61K 31/166; A61P 35/00

(52) U.S. Cl. 514/622

(57) ABSTRACT

The present invention features pharmaceutical compositions that improve the bioavailability of hydrophobic pharmaceutical agents that induce apoptosis in mammalian cells. Also featured are injectable formulations for the parenteral delivery of such hydrophobic pharmaceutical agents into patients in need of such treatment, as well as methods of making and using both the compositions and formulations, including methods for the treatment of cell proliferation or hyperproliferative diseases and disorders.

### RELATED PRIORITY APPLICATION

This application claims priority to U.S. Provisional Application Ser. No. 60/632,335 filed Nov. 30, 2004, the entire content of which is incorporated herein by reference.

### FIELD OF THE INVENTION

The present invention generally relates to pharmaceutical compositions, and particularly relates to liquid pharmaceutical formulations, and methods of making and using the formulations.

### BACKGROUND OF THE INVENTION

The following description of the background of the invention is provided to aid in understanding the invention, but is not admitted to describe or constitute prior art to the invention.

Various methods are available for administering therapeutic compounds to a patient. Such methods include, for example, parenteral, oral, and rectal administration. Variations of these different types of administrations exist. For example, parenteral administration includes intravenous, subcutaneous, intraperitoneal, intramuscular, intrathecal, intramedullary and intratumoral injection. The chosen mode of administration should take into account the nature of the therapeutic compound and the illness that is being treated.

One measure of the potential usefulness of a formulation of a therapeutic compound is the bioavailability of the therapeutic compound observed after parenteral administration of the formulation. Several factors can affect the bioavailability of the therapeutic compound. These factors include aqueous solubility, stability, absorption, and metabolism. Aqueous solubility is one of the most important factors influencing bioavailability. The bioavailability of a therapeutic compound in aqueous solution is generally used as the standard against which other formulations are measured. Formulations that increase the relative bioavailability of the therapeutic compound, as compared to an aqueous solution, are desirable, and, in some cases, critical for the delivery of hydrophobic therapeutic compounds.

Certain potential therapeutic compounds are hydrophobic, having very low aqueous solubility, and thereby exhibiting low bioavailability. Different techniques have been developed for solubilizing hydrophobic therapeutic compounds, such as those described by Schwartz et al., U.S. Pat. Nos. 5,783,592 and 6,335,356, Hausheer et al., U.S. Pat. No. 6,040,330, Chung et al., U.S. Pat. No. 6,046,230, Owens et al., U.S. Pat. No. 6,071,952, and Shenoy et al., U.S. Pat. Nos. 6,248,771 and 6,696,482, all of which are incorporated by reference herein in their entirety.

The instant invention provides pharmaceutical compositions and formulations specifically designed to improve the bioavailability of particular hydrophobic apoptosis-inducing therapeutic compounds. These pharmaceutical compositions and formulations allow for the use of these therapeutic compounds to treat specific diseases and disorders that are associated with the hyperproliferation of cells in an animal.

### SUMMARY OF THE INVENTION

The present invention provides pharmaceutical compositions and formulations for the parenteral administration of the compounds of Formula I (as shown below) to mammals. Compounds of Formula I are potent apoptosis-inducing agents and are particularly effective in treating cancer and other neoplastic diseases associated with the hyperproliferation of cells. In accordance with the present invention, it has been discovered that in general compounds of Formula I are particularly hydrophobic and essentially insoluble in water or other aqueous solutions, such as normal saline. Consequently, the compounds of Formula I have unacceptably low bioavailability when administered directly to mammals.

The present invention provides pharmaceutical compositions and formulations containing the therapeutic compounds of Formula I below with improved bioavailability. Specifically, the present invention provides a liquid pharmaceutical composition suitable for parenteral delivery, particularly intravenous injection, of these compounds. The pharmaceutical composition comprises a solution or in dispersion of an effective amount of one or more compounds of Formula I in one or more semi-solid or liquid solubilizer, preferably non-ionic solubilizer (e.g., surfactants, preferably nonionic surfactants). Optionally, the pharmaceutical composition further comprises a viscosity reducing agent, and/or in admixture with an aqueous diluent. In one specific embodiment, the pharmaceutical composition comprises a solution or dispersion of an effective amount of one or more compounds of Formula I in one or more non-ionic surfactants, and optionally a viscosity reducing agent, and one or more pharmaceutically acceptable aqueous diluent to form an injectable formulation.

The compositions and formulations of the present invention have advantageous characteristics that make possible the administration of hydrophobic pharmaceutical agents of Formula I for both pharmaceutical testing and therapy. In the present invention, these characteristics allow for the parenteral administration, particularly the intravenous injection, of these hydrophobic therapeutic agents. Not only do these formulations overcome the solubility problems shared by the therapeutic agents disclosed, they greatly enhance the bioavailability of the agents in test animals.

Thus, a first aspect of the present invention entails preparing a primary formulation comprising: (a) one or more hydrophobic pro-apoptotic therapeutic agents (i.e., compounds of Formula I); (b) one or more non-ionic solubilizing agents, particularly surfactants, and especially non-ionic surfactants; and, optionally, (c) one or more viscosity reducing agents. Another aspect of the present invention entails preparing an injectable formulation of the hydrophobic pro-apoptotic agents of Formula I by combining the primary formulation with a pharmaceutically acceptable aqueous diluent, such as WFI (water for injection), D5W (5% dextrose in water), normal saline, or lactated Ringer's solution. Yet another aspect of the present invention provides kits for preparing injectable formulations of the hydrophobic pro-apoptotic agents of Formula I. Finally, still another aspect of the present invention entails methods of using the injectable formulations of hydrophobic pro-apoptotic agents of Formula I for the treatment of patients in need of such therapy, particularly for the treatment of patients with diseases and disorders, such as, cancer and other neoplastic diseases, that are associated with the hyperproliferation of cells within the body of an individual. Such diseases and disorders will be referred to herein as “hyperproliferative” diseases and disorders.

Alternative embodiments of both the primary formulation and injectable formulation are described. In all embodiments it is anticipated that the one or more hydrophobic pro-apoptotic agents used in these formulations will include one or more compounds of Formula I, as described above. Additional embodiments encompassing formulations that combine the compounds of Formula I with other bioactive molecules, and especially other cancer chemotherapeutic agents, are envisioned and described. Advantageously, such formulations can be used in the treatment of diseases and disorders involving abnormal or undesired cell proliferation, such as cancers and neoplastic diseases, as well as other hyperproliferative diseases and disorders.

Other features and advantages of the invention will be apparent from the following description of the alternative aspects and embodiments of the invention, and from the claims listed below.

### BRIEF DESCRIPTION OF THE FIGURES

FIG. 1 shows the phase solubility of 5-Chloro-N-[2-(4-chloro-naphtalen-1-yloxy)-5-trifluoromethyl-phenyl]-2-hydroxy-benzamide solubilized with surfactants or a complexing agent, and added to 5% dextrose in water (D5W);

FIG. 2 shows the stability of 5-Chloro-N-[2-(4-chloro-naphtalen-1-yloxy)-5-trifluoromethyl-phenyl]-2-hydroxy-benzamide solubilized with Cremophor EL:Ethanol (1:1) [a pharmaceutical composition of the present invention], at elevated temperature (60° C.); and

FIG. 3 shows the stability of 5-Chloro-N-[2-(4-chloro-naphtalen-1-yloxy)-5-trifluoromethyl-phenyl]-2-hydroxy-benzamide solubilized with Cremophor EL:Ethanol (1:1) and diluted 9-fold with D5W [an injectable formulation of the present invention], at room temperature.

### DETAILED DESCRIPTION OF THE INVENTION

The present invention features pharmaceutical compositions of the therapeutic compounds of Formula I.

In a first aspect of the present invention, pharmaceutical compositions and methods of preparing such pharmaceutical compositions are provided. Such compositions comprise a solution or dispersion of at least one hydrophobic therapeutic compound of Formula I in a semi-solid or liquid nonionic solubilizer, particularly nonionic surfactant. Such compositions can optionally contain other components, such as viscosity reducing agents, preservatives, antioxidants, pH-adjusting compounds, osmolarity-adjusting compounds, stabilizers and any other components, so long as those components do not adversely affect the pharmaceutical acceptability of the final composition, or the bioavailability of the solubilized therapeutic compounds.

In preferred embodiments of this aspect of the invention, the therapeutic compounds of Formula I are fully solubilized by a solution comprising a pharmaceutically acceptable surfactant and, optionally, a viscosity reducing agent. When included, the viscosity reducing agent serves to reduce the viscosity of the pharmaceutical composition sufficiently enough to allow for convenient handling by syringe (syringability) and/or sterilization by filtration (filterability).

In one embodiment, the pharmaceutical composition of the present invention is in a concentrated form, herein referred to as “primary formulation,” which either is substantially free of an aqueous diluent or has an insufficient amount of an aqueous diluent for it to be directly injectable intravenously into a mammal.

In another embodiment, the pharmaceutical composition comprises a sufficient amount of a pharmaceutically acceptable aqueous diluent such that the pharmaceutical composition is directly injectable, particularly intravenously, to a mammal. This form of the pharmaceutical composition is referred to herein as “injectable formulation.” Thus, methods are provided for the preparation of the sterile injectable formulations of the instant invention; formulations to be administered parenterally, particularly by intravenous injection, to patients in need of such treatment. In this aspect of the invention, a concentrated primary formulation is combined with a pharmaceutically acceptable aqueous diluent, such as WFI (water for injection), D5W (5% dextrose in water), normal saline, and lactated Ringer's solution, etc., in order to prepare the injectable formulation that is subsequently administered to the patient.

The amount of primary formulation used in the preparation of an injectable formulation can advantageously be adjusted to provide a therapeutically effective dose of the therapeutic compound of Formula I in the injectable formulation, which can then be administered to the patient. The effective dose can be adjusted to account for such variables as the mass and relative health of the patient in need of treatment. As mentioned above, the primary formulation used in the preparation of the injectable formulation, being of sufficiently low viscosity to allow for ready sterile filtration, can advantageously be sterile filtered before it is combined, or as it is combined, with a sterile pharmaceutically acceptable aqueous diluent to make a sterile injectable formulation for administration to a patient.

In another aspect of the present invention, kits are provided for use in preparing the injectable formulation for administration to patients, or in preparing the primary formulation in addition to the injectable formulation.

In yet another aspect, the present invention provides methods of treating, or delaying the onset of, symptoms of an abnormal condition involving the proliferation or hyperproliferation of cells in a patient in need of such treatment. The method comprises the following steps: (a) identifying a patient in need of such treatment; and (b) parenterally administering the injectable formulation to the patient. Optionally, the patient is premedicated with a medicament that reduces or eliminates hypersensitivity reactions before treated with the injectable formulation of the present invention.

### Pro-Apoptotic Therapeutic Compounds

Thus, in accordance with the present invention, a pharmaceutical composition is provided comprising in solution or dispersion a therapeutically effective amount of a compound of Formula I

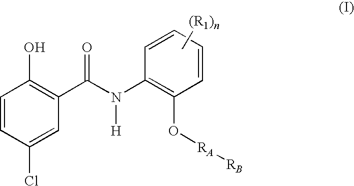

wherein

R_{A} is a bond, or a straight chain or branched C₁₋₆ alkyl;

R_{B} is, a phenyl group optionally substituted with 1-6 halo or C₁₋₆ haloalkyl substituents, or a napthyl group optionally substituted with 1-7 halo or C₁₋₆ haloalkyl substituents;

R₁ is, independently, halo or C₁₋₆ haloalkyl, and n is an integer from 0 to 4, in a semi-solid or liquid nonionic solubilizer. Preferably the solubilizer is a pharmaceutically acceptable nonionic surfactant in a sufficient amount to dissolve said compound and to maintain said compound in solution or dispersion when mixed with a pharmaceutically acceptable aqueous solution (i.e., diluent), as required for parenteral delivery.

The compounds of Formula I are potent apoptosis-inducing agents and are particularly effective in treating diseases and disorders involving abnormal or undesired cell proliferation, such as cancer and neoplastic diseases, and other hyperproliferative diseases and disorders. However, it has been recognized that when administered orally to animals, the compounds are not absorbable to any meaningful extent. Moreover, the compounds are substantially insoluble in aqueous solutions, and thus are not, on their own, suitable for injection via aqueous carriers. In other words, the compounds of Formula I cannot be effectively administered directly.

The compositions and formulations of the present invention facilitate solubilization and delivery of the hydrophobic pharmaceutical agents of Formula I, which, by themselves, are only weakly soluble in aqueous solutions, but which readily dissolve in non-ionic surfactants and various organic solvents.

In one set of embodiments, the therapeutic compounds in the compositions of the present invention are according to Formula (II):

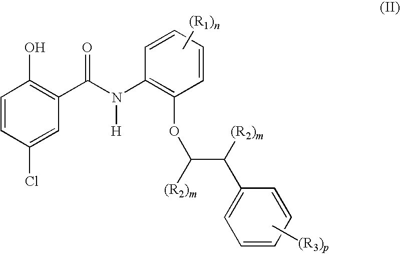

wherein

R₁ is, independently, halo (preferably Cl or F) or C₁₋₆ haloalkyl (preferably trifluoromethyl), and q is an integer from 0 to 4.

R₂ is, independently, straight chained or branched C₁₋₄ alkyl, and m=0-2; and

R₃ is, independently, halo or C₁₋₆ haloalkyl, and p=0-5.

In another set of embodiments, the therapeutic compounds in the compositions of the present invention are according to Formula (III):

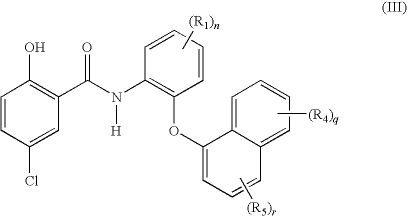

wherein

R₁ is, independently, halo (preferably Cl or F) or C₁₋₆ haloalkyl (preferably trifluoromethyl), and n is an integer from 0 to 4;

R₄ is, independently, halo or C₁₋₆ haloalkyl, and n=0-4; and

R₅ is, independently, halo or C₁₋₆ haloalkyl, and n=0-3.

In specific embodiments, the therapeutic compounds in the compositions of the present invention are selected from:

5-Chloro-N-{2-[2-(4-chloro-phenyl)-3-methyl-butoxy]-5-trifluoromethyl-phenyl}-2-hydroxy-benzamide:

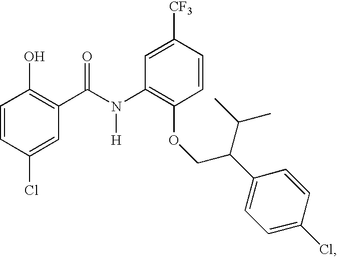

5-Chloro-N-{5-chloro-2-[2-(4-trifluoromethyl-phenyl)-ethoxy]-phenyl}-2-hydroxy-benzamide:

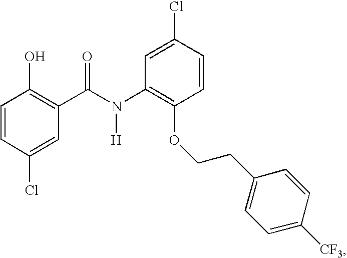

5-Chloro-N-{4-chloro-2-[2-(4-chloro-phenyl)-ethoxy]-phenyl}-2-hydroxy-benzamide:

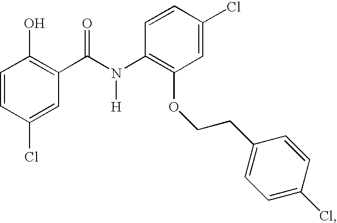

5-Chloro-N-{2-[2-(3,4-dichloro-phenyl)-ethoxy]-5-trifluoromethyl-phenyl}-2-hydroxy-benzamide:

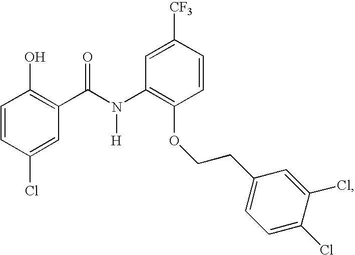

5-Chloro-N-[2-(4-chloro-naphtalen-1-yloxy)-5-trifluoromethyl-phenyl] -2-hydroxy-benzamide:

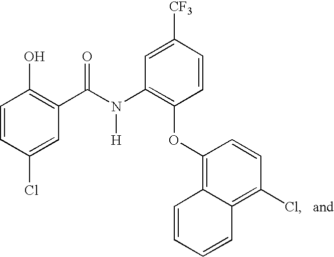

5-Chloro-N-[5-chloro-2-(4-chloro-naphthalen-1-yloxy)-phenyl]-2-hydroxy-benzamide:

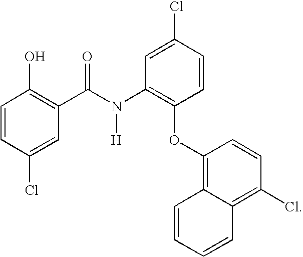

Additionally, the pharmaceutical compositions of the instant invention may also include other therapeutic compounds or agents, which are to be co-administered with the therapeutic compounds of Formula I. These other therapeutic compounds may include agents of any pharmaceutical class, so long as their inclusion does not severely compromise, or otherwise adversely affect, the therapeutic value of the compounds of Formula I. Hence, it is anticipated that the one or more hydrophobic pharmaceutical agents present in the pharmaceutical compositions of the instant invention can include a combination of a compound, or compounds, of Formula I, combined with one or more other anti-cancer agents, i.e., other compounds effective in the treatment of cell proliferation or hyperproliferative disorders or diseases. In particular, the one or more hydrophobic pharmaceutical agents provided by Formula I may be formulated in combination of at least one known cancer chemotherapeutic agent, or a pharmaceutically acceptable salt or prodrug of said agent. Examples of known cancer chemotherapeutic agents which may be used for combination therapy include, but not are limited to, alkylating agents, antimitotic agents, topo I inhibitors, topo II inhibitors, RNA/DNA antimetabolites, DNA antimetabolites, EGFR inhibitors, proteosome inhibitors, angiogenesis inhibitors, estrogen receptor antagonists, etc.

### Pharmaceutical Compositions

The inventors of the present invention have discovered novel pharmaceutical compositions in which the compounds of Formula I are substantially soluble (in solution or in dispersion) at a sufficiently high concentration. Specifically, the concentrated pharmaceutical composition, i.e., the primary formulation is substantially stable as a solution or dispersion under normal handling and storage conditions. As such these compositions containing compounds of Formula I can be used to prepare the injectable formulations of the present invention that can be parenterally administered to mammals, and show significantly improved bioavailability and/or resorption. The invention thus enables parenteral administration of the therapeutic compounds of Formula I at a lower dosage level to achieve effective therapy.

### 1. Primary Formulation

Thus, a first aspect of the instant invention provides a concentrated pharmaceutical composition, i.e., primary formulation, comprising: (a) an effective amount of one or more hydrophobic therapeutic agents, where the agents are compounds of Formula I and (b) one or more pharmaceutically acceptable semi-solid or liquid nonionic solubilizer, preferably nonionic surfactants and, optionally, (c) one or more pharmaceutically acceptable viscosity reducing agents. The compound(s) are in solution or dispersion in the surfactant(s) and/or viscosity reducing agent(s).

Alternatively, this aspect of the present invention features a concentrated pharmaceutical composition, i.e., primary formulation, comprising: (a) a therapeutically effective amount of at least one therapeutic compound of Formula I; and (b) a solution or “liquid vehicle” further comprising a pharmaceutically acceptable surfactant and, optionally, a viscosity reducing agent in a ratio of about 10:1 to about 1:10 (v/v). In this alternative first aspect of the invention, the therapeutic compound of Formula I is said to be solubilized by the liquid vehicle whereupon it forms a stable solution or dispersion that is the pharmaceutical composition.

The term “liquid vehicle,” as used herein, means a pharmaceutically acceptable substance that is a liquid at about room temperature to about 37° C., comprising one or more components approved for pharmaceutical use, and which is capable of dissolving the therapeutic compounds of Formula I to form a true solution or an emulsion. Pharmaceutically acceptable liquid vehicles of the instant invention can comprise a surfactant alone, or a surfactant in combination with other components, such as viscosity reducing agents, aqueous diluents, preservatives, antioxidants, pH adjustment agents, osmolarity adjustment agents and stabilizers. Advantageously, the pharmaceutically acceptable liquid vehicles of the instant invention are capable of solubilizing or dissolving the therapeutic compounds of Formula I forming a substantially stable solution or dispersion, which when in admixture with an aqueous diluent, form a solution or dispersion suitable for intravenous administration into a patient. That is, the pharmaceutically acceptable liquid vehicles of the instant invention are capable of keeping the therapeutic compounds of Formula I in solution in such aqueous solutions before and during their administration to the patient.

The amount of therapeutic compound of Formula I per unit volume of the primary formulation can be greater than that in the injectable formulations, which are made with it. In certain embodiments of the instant invention, the concentration of therapeutic compound of Formula I in the concentrated pharmaceutical composition, i.e., primary formulation is between about 1 mg/ml and about 50 mg/ml. In certain specific embodiments, the concentration of compound is between about 5 mg/ml and about 10 mg/ml. Specially, In the primary formulation of the instant invention the ratio (weight/volume, i.e., w/v) between the therapeutic compound(s) and surfactant(s) is preferably from about 1 mg/L to about 500 g/L, more preferably from about 1 g/L to about 300 g/L.

Optionally, the primary formulation of the present invention may contain other components, as required, to enhance the handling characteristics, the stability of the composition, or the bioavailability of the therapeutic compounds of Formula I. These other components particularly include viscosity reducing agents, as well as preservatives, antioxidants, pH adjusting compounds, osmolarity adjusting compounds and stabilizers.

Any solubilizers known in the art can be used so long as when present in a sufficient amount and in admixture with the therapeutic compound of Formula I, the solubilizer can dissolve or disperse the therapeutic compound of Formula I and to maintain it in solution or dispersion when mixed with a pharmaceutically acceptable aqueous solution (i.e., diluent), as required for parenteral delivery. As is apparent to a skilled artisan, solubilizers include surfactants, co-solvents, and complexing agents.

In preferred embodiments, the solubilizer is a surfactant. As is known in the art, surfactants are amphipathic molecules comprised of a hydrophobic part and a hydrophilic part, and can be anionic, cationic, amphoteric or non-ionic, the surfactants. In preferred embodiments, a “pharmaceutically acceptable nonionic surfactant” is employed, which is capable of (a) dissolving the therapeutic compound of Formula I, (b) forming micelles when introduced into a pharmaceutically acceptable aqueous diluent, and (c) keeping the therapeutic compounds solubilized or dissolved in such aqueous solutions before and during their administration to the patient.

Generally, surfactants are discussed in detail in the book, Surfactants Systems, Their Chemistry, Pharmacy and Biology, by D. Attwood and A. T. Florence, (Chapman and Hall Pub. Co., 1983), which is incorporated herein by reference in its entirety. Relatively common examples of surfactants include potassium laurate, sodium alkylsulfates such as sodium dodecyl sulfate, hexadecyl sulphonic acid, and sodium dioctylsulphosuccinate, hexadecyl(cetyl)trimethylammonium bromide, dodecylpyridinium chloride, dodecylamine hydrochloride, N-dodecyl-N,N-dimethyl betaine, bile acids and salts, acacia, tragacanth, Igepal (polyoxyethylated nonylphenols), sorbitan esters (Spans), polysorbates (Tweens), Triton-X analogs (polyoxyethylated t-octylphenols), Brij analogs selected from the group consisting of polyoxyethylene lauryl ethers, polyoxyethylene cetyl ethers, polyoxyethylene stearyl ethers, and polyoxyethylene oleyl ethers, Myrj analogs (polyoxyethylene stearates), pluronics and tetronics selected from the group consisting of poloxamer and poloxamine type polyoxyethylene-polyoxypropylene derivatives, surface active drug agents such as phenothiazines and tricyclic antidepressants, and the like. Surfactants can be selected from the list above. Preferably, semi-solid or liquid non-ionic surface active agents, i.e., surfactants are used, especially esters and ethers of polyoxyalkene glycols, esters and ethers of polyhydric alcohols, or esters and ethers of phenols. Poloxamers and poloxamines are also useful. Semi-solid or liquid non-ionic surfactants are preferably chosen from polyethoxylated fatty acids, hydroxylated fatty acids and fatty alcohols. Specific preferred examples include, but are not limited to, polyoxyethylene castor oil derivatives. In certain embodiments, the nonionic surfactant is a polyethoxylated castor oil, polyethoxylated hydrogenated castor oil, polyethoxylated fatty acid from castor oil or polyethoxylated fatty acid from hydrogenated castor oil. In specific embodiments the polyethoxylated castor oil is Cremophor. In certain specific embodiments, the polyethoxylated castor oil is Cremophor EL® (BASF, Ludwigshafen, Germany). In other embodiments the non-ionic surfactant is Incordas 30, polyoxyethylene 5 castor oil, polyethylene 9 castor oil, polyethylene 15 castor oil, polyoxyl-15-hydroxystearate which is also known as 12-hydroxystearic acid-polyethylene glycol copolymer (Solutol HS-15), d-alpha-tocopheryl polyethylene glycol succinate (TPGS), or monoglycerides, such as myverol, or aliphatic alcohol based nonionic surfactants, such as oleth-3, oleth-5, polyoxyl 10 oleyl ether, oleth-20, steareth-2, stearteth-10, steareth-20, ceteareth-20, polyoxyl 20 cetostearyl ether, PPG-5 ceteth-20, and PEG-6 capryl/capric triglyceride, Pluronic® copolymer non-ionic surfactants, such as Pluronic® L10, L31, L35, L42, L43, L44, L62, L61, L63, L72, L81, L101, L121, and L122, sorbitan fatty acid esters, such as Tween 20, Tween 40, Tween 60, Tween 65, Tween 80, Tween 81, and Tween 85, or, finally, ethoxylated glycerides, such as PEG 20 almond glycerides, PEG-60 almond glycerides, PEG-20 corn glycerides, and PEG-60 corn glycerides.

As suggested above, in the instant invention, surfactants are chosen partly for their ability to solubilize the compounds of Formula I, and partly for their ability to form micelles when introduced into pharmaceutically acceptable aqueous diluents, in which the compounds of Formula I remain dissolved or solubilized or dispersed. Micelles are microscopic spherical arrangements of clusters of amphipathic organic molecules, such as surfactants, which are formed in aqueous solutions by the propensity of the hydrophobic parts of individual amphipathic molecules to cluster and be buried inside the sphere, from which water is excluded, and the hydrophilic parts to remain on the surface of the sphere where they remain in contact with water. The propensity of surfactants to form micelles is determined by the structure of the individual surfactant molecules and the nature of their hydrophobic and hydrophilic parts.

Importantly, surfactants are frequently characterized by a physical property known as the critical micelle concentration, or CMC. The CMC is a measure of the ability of surfactants to form micelles when introduced into aqueous solutions. Specifically, the CMC is the lowest concentration of a surfactant at which the surfactant molecules self-associate with each other to form micelles. Concentrations of surfactant that are greater than the CMC are also attended by micellization, with more complex self-associated structures forming as the surfactant concentration is increased further. The CMC value is also referred to as a critical value, because it represents a threshold, below which micelles cannot form. Values of CMCs are generally expressed in percent, representing the fractional concentration of the amphipathic molecule (i.e., surfactant) in the aqueous solution, above which micelles can form. Additionally, depending upon the relative strengths of the hydrophilic and hydrophobic interactions of the amphipathic surfactant molecules, and the temperature of the solution, the CMC is the minimum concentration (in fractional percent) needed for micelles to be stable once formed.

In the instant invention, the preferred non-ionic surfactants have a CMC number in the range of about 0.005% to about 0.50%, preferably about 0.01% to about 0.10%, more preferably about 0.01% to about 0.05%. Preferably, the non-ionic surfactant used to dissolve the compounds of Formula I have an CMC number of between about 0.01% to about 0.10%, preferably about 0.01% to about 0.05%. Non-ionic surfactants with CMC numbers in this range have been found to provide the preferred combination of physical characteristics, both in terms of solubilizing the compounds of Formula I, and in forming micelles when the pharmaceutical composition is introduced into a larger volume of pharmaceutically acceptable aqueous diluent.

Surfactants are also categorized and characterized by their hydrophilicity-lipophilicity balance number, or “HLB number.” The HLB number is a number on the scale of one to 40, according to the HLB system introduced by Griffin (See Surfactants Systems, Their Chemistry, Pharmacy and Biology, by D. Attwood and A. T. Florence, (Chapman and Hall Pub. Co., 1983), which is incorporated herein by reference). The HLB system is a semi-empirical method used to predict what type of surfactant properties a particular molecular structure will provide. The HLB system is based on the concept that some molecules have hydrophilic groups, other molecules have lipophilic groups, and some have both. Weight percentage of each type of group on a molecule, or in a mixture, predicts what behavior the molecular structure, or composition, will exhibit. Consequently, with respect to surfactants, the HLB number is a semi-empirical measure of the emulsifying power of the surfactant that corresponds to the proportion of hydrophilic groups and lipophilic groups on the surfactant molecule.

In practical use, the HLB number predicts the behavior of the surfactant, with respect to its “water-loving” or “lipid-loving” nature, and its ability to solubilize organic molecules. In particular, the higher the number, the more hydrophilic the surfactant, and the lower the number the more lipophilic the surfactant. The HLB number required for solubilizing a particular organic compound or drug is determined empirically by selecting a surfactant with a known HLB number, blending it with the compound or drug and observing the solubilization results. A true solution of the compound or drug is formed when a surfactant with an appropriate HLB number is used, while a non-uniform mixture indicates a surfactant with a different HLB number is needed to properly solubilize the compound or drug.

The HLB number of different surfactants can be used as a guide in the selection of a surfactant suitable for solubilizing a particular compound. The HLB numbers for many surfactants is generally known in the art, and can also be experimentally determined. Furthermore, HLB numbers are algebraically additive. Thus, by combining a surfactant with a low HLB number with a surfactant with a high HLB number, mixtures of surfactant can be prepared that exhibit HLB numbers intermediate between the two HLB numbers of the starting surfactants. The concept of HLB numbers is detailed in Remington's Pharmaceutical Sciences, 16th Ed., Mack Pub. Co., (1980), pages 316-319.

In preferred embodiments, non-ionic surfactants with HLB numbers in the range of about 10 to about 16 are used. Preferably, the non-ionic surfactant used to dissolve or disperse the compounds of Formula I has an HLB number of between about 12 to about 14. Non-ionic surfactants with HLB numbers in this range have been found to provide the preferred combination of physical characteristics, both in terms of solubilizing the compounds of Formula I, and forming micelles when added to pharmaceutically acceptable aqueous diluents.

Examples of non-ionic surfactants that can be used in preparing the formulations of the instant invention particularly include the polyethoxylated castor oils. The term “ethoxylated castor oil,” as used above and herein, refers to castor oil that is modified with at least one oxygen-containing moiety. In particular the term refers to castor oil comprising at least one ethoxyl moiety. Furthermore, as used herein, the term polyoxyl 35 castor oil, which is also known as PEG-35 Castor Oil, Macrogoglycerol ricinoleate and Macrogoglyceroli ricinoleas, and alternatively as CAS Registry No. 61791-12-6, is a non-ionic surfactant, solubilizer and emulsifying agent used in the aqueous formulation of hydrophobic substances. Polyoxyl 35 castor oil is prepared by reacting castor oil with ethylene oxide in a molar ratio of 1:35. “Cremophor EL®” (BASF, Ludwigshafen, Germany) is a polyoxyl 35 castor oil which has an HLB number between 12 and 14, and a critical micelle concentration (CMC) of approximately 0.02%. Cremophor EL® has a density at 25° C. of 1.05-1.06 g/ml, and a viscosity of 700-800 mPa·s (See Product Literature on Cremophor EL® from BASF).

Other non-ionic surfactants that can be used to prepare the compositions and formulations of the present invention include various forms of polysorbates (e.g., Tween-80), sorbitan esters (e.g., Spans), Brij analogs, (e.g., polyoxyethylene lauryl ethers, polyoxyethylene cetyl ethers, polyoxyethylene stearyl ethers, and polyoxyethylene oleyl ethers), and Myrj analogs (polyoxyethylene stearates), pluronics and tetronics selected from the group consisting of poloxamer and poloxamine type polyoxyethylene-polyoxypropylene derivatives. Other examples of suitable surfactants include polyoxyl-15-hydroxystearate which is also known as 12-hydroxystearic acid-polyethylene glycol copolymer (Solutol HS-15), POLYSORBATE 80® and other polyoxyethylene sorbitan fatty acid esters, glyceryl monooleate, polyvinyl alcohol, ethylene oxide copolymers such as PLURONIC® (a polyether), polyol moieties, and sorbitan esters. In preferred embodiments ethoxylated castor oils, such as Cremophor® EL, are used for the formulation of the pro-apoptotic therapeutic agents of Formula I.

Beneficially, the surfactants used in preparing the composition of the instant invention allow the therapeutic compounds of Formula I to be solubilized or dispersed in, and delivered by way of, an aqueous diluent. Such formulations are designed for delivery by parenteral routes, especially via intravenous injection.

### Viscosity Reducing Agents:

In further accordance with the present invention, the pharmaceutical compositions provided may additionally include one or more viscosity reducing agents. As used herein, the term “viscosity reducing agent” means a suitable compound that, when mixed with a surfactant, or included in a liquid vehicle, reduces the viscosity of the surfactant or liquid vehicle to such an extent that the resulting solution can be readily handled by syringes and can be readily sterile filtered. Advantageously, viscosity reducing agents of the instant invention reduce the viscosity of the solubilizer, especially the surfactant, or liquid vehicle to the point where the resulting solutions can be filtered through sterile filters bearing pores of 0.22 micrometers (μm), or less at room temperature. Such viscosity reducing agents allow for the use of semi-solid or liquid surfactants that, by themselves, are too viscous to be readily handled by syringes or sterile filtered, in the compositions and formulations of the instant invention. They also therefore improve the handling characteristics of the liquid vehicle used to reconstitute the therapeutic compounds of Formula I.

As used herein, the term “syringability” or “syringable” means the ability of a solution or dispersion to be handled conveniently and accurately by a syringe fitted with a hypodermic needle of no greater diameter than a 15 gauge needle at room temperature. Furthermore, “syringable solutions” can be readily handled, and volumetrically measured by means of a hypodermic syringe.

The term “filterability” or “filterable” as used herein, means the ability of a solution or dispersion to be passed through a filter medium, and in the instant situation, means the ability of a solution to be readily passed through a filter with no greater than 0.22 micrometer (μm) pores, to allow for the sterilization of the solution by the process of filtration at room temperature. In particular, sterile filtration of the compositions and formulation of the instant invention can be achieved by passing compositions and formulations through a “sterile filter” with a pore size of 0.22 micrometer (μm), or less. For example, sterile filtration of the compositions and formulation of the instant invention can be achieved by passing these solutions through a polyvinylidene fluoride (PVDF) membrane with a pore size of 0.22 micrometer (μm), such as that found in “Durapore”™ filters (Millipore, Billerica, Mass., USA).

In certain embodiments of the instant invention, such as those embodiments where the non-ionic surfactant used in the primary formulation is a polyethoxylated castor oil, a viscosity reducing agents is preferably included in the concentrated composition to allow for convenient handling of solubilized or dispersed therapeutic compounds of Formula I. In certain embodiments, this is preferred because the non-ionic surfactant employed is a semi-solid or paste, and not a liquid, at room temperature. In these embodiments, the viscosity reducing agent and the non-ionic surfactant are preferably premixed before the addition of a therapeutic compound of Formula I. The volume/volume ratio of non-ionic surfactant to viscosity reducing agent can be adjusted so as to prepare a mixture that is a liquid at room temperature. Preferably, this liquid mixture will be of sufficiently low viscosity that it is syringable or filterable. In these embodiments, the therapeutic compound of Formula I is solubilized in the liquid mixture of non-ionic surfactant and viscosity reducing agent. Such a liquid mixture is referred to as a “liquid vehicle.”

In those embodiments of the instant invention where a viscosity reducing agent is included in the pharmaceutical composition of the invention, the volume of viscosity reducing agent present in the composition, relative to the volume of surfactant, particularly non-ionic surfactant can be from about 1:10 to about 10:1. In certain embodiments, the relative volumes are from about 1:2 to 2:1. In some of these embodiments, the relative volumes are about 1:1. However, as a skilled artisan would recognize, the ratio between viscosity reducing agent and surfactant may vary, depending on the surfactant and viscosity reducing agent used. Regardless, viscosity reducing agent and surfactant are used in a ratio to achieve a viscosity such that the primary formulation is syringable or filterable.

Examples of viscosity reducing agents that can be used in preparing the formulations of the instant invention include the C₁₋₅ alkanols (such as ethanol, n-propanol and isopropanol), the monoesters of glycerol (e.g., glycerol monocaprylate and glycerol monooleate), as well as aliphatic mono carboxylic acids that are liquids at room temperature and above. Exemplary viscosity reducing agents that may be used in the pharmaceutical composition of the present invention include, but are not limited to, alcohols such as ethanol or isopropanol, n-propyl alcohol, polyoxyethylene 5 castor oil, polyoxyethylene 9 castor oil, labrafil, labrasol, capmul GMO (glyceryl mono oleate), capmul MCM (medium chain mono- and diglyceride), capmul MCM C8 (glyceryl mono caprylate), capmul MCM C10 (glyceryl mono caprate), capmul GMS-50 (glyceryl mono stearate), caplex 100 (propylene glycol didecanoate), caplex 200 (propylene glycol dicaprylate/dicaprate), caplex 800 (propylene glycol di 2-ethyl hexanoate), captex 300 (glyceryl tricapryl/caprate), captex 1000 (glyceryl tricaprate), captex 822 (glyceryl triandecanoate), captex 350 (glyceryl tricaprylate/caprate/laurate), caplex 810 (glyceryl tricaprylate/caprate/linoleate), capmul PG8 (propylene mono caprylate), propylene glycol, and propylene glycol laurate (PGL).

The pharmaceutical compositions of the present invention can optionally contain other components, such as preservatives, antioxidants, pH adjusting compounds, osmolarity adjusting compounds, stabilizers and any other components, so long as those components do not adversely affect the pharmaceutical acceptability of the final composition, or the bioavailability of the solubilized therapeutic compounds.

In particular, the compositions of the present invention may contain pharmaceutically acceptable preservatives.

Preservatives are generally viewed as agents that prevent of inhibit microbial growth in a formulation. Common preservatives are the parabens (e.g. methyl, ethyl, propyl, and butyl paraben), ethanol and isopropanol, sodium benzoate, benzyl alcohol, chlorobutanol, phenol, potassium sorbate, thimerosal, benzalkonium chloride.

Additionally, the compositions of the present invention may contain pharmaceutically acceptable antioxidants. Such antioxidants serve to protect the components of the compositions from oxidative damage caused by molecular oxygen or reactive oxygen species. Examples of pharmaceutically acceptable antioxidants that can be included in the compositions of the present invention include, for example, ascorbic acid, sodium ascorbate, ascorbyl plamitate, BHA (butylated hydroxyanisole), BHT (butylated hydroxytoluene), vitamin E, vitamin E PEG 1000, TPGS, and the like. Also, the compositions of the present invention may contain pharmaceutically acceptable pH adjusting compounds and/or osmolarity adjusting compounds. Such compounds are used to improve the characteristics for the pharmaceutical composition so that it can be used to prepare formulations that are optimized for parenteral administration, especially intravenous injection. Examples of suitable pH adjusting compounds include any pharmaceutically acceptable buffering system (e.g., phosphate, acetate, carbonate, tromethamine, citrate, lactate), or any acceptable acidifying (e.g., hydrochloric acid, tartaric acid, acetic acid, citric acid) or alkalizing (sodium or potassium hydroxide, monoethanolamine, diethanolamine, triethanolamine) agents. Examples of suitable osmolarity adjusting compounds include any pharmaceutically acceptable water soluble compound, either ionic or nonionic in nature, for example, glucose, sucrose, fructose, sodium chloride, sodium lactate, sorbitol, mannitol, glycerin, polyethylene glycols 400 to 4000, and all pharmaceutically acceptable buffer salts.

### Methods of Preparing Pharmaceutical Compositions

Solubilizing or dispersing the compounds of Formula I in a solubilizer can be accomplished by a variety of techniques known to those skilled in the art. These techniques include stirring techniques (manually and with magnetic stirring systems), vortexing techniques, vibration techniques, and sonication techniques. In certain embodiments, the compounds of Formula I may be dissolved in a cosolvent before being combined with a non-ionic surfactant. Advantageously, that cosolvent can also serve as a viscosity reducing agent. In specific embodiments, the cosolvent is a C₁₋₅ alkanol, such as ethanol. In other embodiments, the compounds of Formula I are dissolved or solubilized directly in a liquid vehicle comprising a nonionic surfactant and, optionally, a viscosity reducing agent. The concentration of therapeutic compound of Formula I in the pharmaceutical compositions of the present invention can be conveniently adjusted, as required, during preparation of the compositions by altering the mass of compound added to a fixed volume of liquid vehicle. Alternatively the concentration may be adjusted by altering the volume of liquid vehicle used to solubilize a fixed mass of compound. In either case, the combination is mixed until uniform.

If necessary, the viscosity of the mixture can be adjusted, or further adjusted, by adding a viscosity reducing agent. Once the desired composition (and viscosity) is obtained, the composition can be sterile-filtered and aliquoted as required.

It is worth noting that the pharmaceutical compositions of the present invention can be prepared in bulk before delivery to clinics responsible for administering the compositions to patients in need of such treatment. Alternatively, the pharmaceutical compositions of the present invention can be prepared at such clinics, immediately before administration to the patients. Either way, the amount of therapeutic compound of Formula I administered to a patient can be conveniently controlled by adjusting the amount of the pharmaceutical composition used to make the injectable formulation.

### 2. Injectable Formulations

Another aspect of the instant invention is an injectable formulation which comprises in solution or dispersion an effective amount of a therapeutic compound of Formula I in a semi-solid or liquid solubilizer in admixture with an aqueous diluent. The injectable formulation can be prepared by mixing a primary formulation described above with an aqueous diluent.

In a preferred embodiment, the injectable formulation comprises one or more hydrophobic pharmaceutical agents, where the agents are compounds of Formula I and, one or more pharmaceutically acceptable surfactants, and, optionally, one or more pharmaceutically acceptable viscosity reducing agents, mixed with a pharmaceutically acceptable aqueous diluent. While not wishing to be bound by any theory, it is believed that in the injectable formulations of the instant invention, the therapeutic compounds of Formula I are localized within self-associated surfactant structures, including micelles, which are formed by the surfactants used in preparing the pharmaceutical compositions of the instant invention when contact is made with water. As such, the therapeutic compounds of Formula I can be delivered into a patient in need of treatment, by means of parenteral administration, and, in particular, by intravenous injection or infusion of the formulation.

Any suitable aqueous diluent known in the art can be used. Examples of pharmaceutically acceptable aqueous diluents include the solutions commonly used to prepare solutions for intravenous administration, and include, among other things, “water for injection” (WFI), 5% dextrose (glucose) in water (D5W), normal saline, 5% dextrose in ½ normal saline (D5W ½ N saline), and lactated Ringer's solution. Advantageously, when a concentrated composition or primary formulation of the instant invention having a non-ionic surfactant is mixed with a pharmaceutically acceptable aqueous diluent, the compositions form self-association structure, including micelles, that keep the therapeutic compound of Formula I in solution before and during administration of the resulting injectable formulation to patients.

Typically in the injectable formulation of the present invention, the ratio (volume to volume) between the solubilizer, preferably non-ionic surfactant and the aqueous diluent is from about 0.01:500 to about 1:1 (v/v), more preferably about 1:500 to about 1:2 (v/v), and most preferably about 1:200 to 1:5 (v/v). A skilled artisan would recognize that the ratio may vary with the solubilizer or surfactant and the aqueous diluent used, so long as the final injectable formulation is “metastable” and “injectable.”

The term “injectable,” as used herein, means suitable for injection into a patient via a syringe with a 15 gauge needle, particularly by parenteral delivery within the veins of a patient.

The term “metastable” as used herein, means either a true solution, or an emulsion or microemulsion or dispersion that remains physically and chemically stable for a period of time before it begins to change in character. In the instant case, the metastable solution formed upon addition of a concentrated pharmaceutical composition to a pharmaceutically acceptable aqueous diluent must remain stable at temperatures of about 20 to about 37° C. for a long enough period of time to allow for delivery of a therapeutically effective amount of a compound of Formula I, solubilized in that solution, or an emulsion or microemulsion or dispersion to be administered to a patient. Preferably, it is stable at at temperatures of about 20 to about 37° C. for at least about 1 hour, at least about 4 hours, more preferably about 8 hours, even more preferably about 16 hours, and still even more preferably about 24 or more hours.

Typically the injectable formulations of the present invention are prepared by diluting an aliquot of the concentrated pharmaceutical composition in a pharmaceutically acceptable aqueous diluent. Advantageously, the concentrated pharmaceutical composition or primary formulation may be volumetrically measured and transferred by syringe, and the transferring may include a sterilization step in which the pharmaceutical composition is passed through a suitable sterile filtration device with a filtration pore size of 0.22 μm, or less. In certain embodiments of the present invention, the pharmaceutical composition is passed through a sterile filtration device as it is being delivered into a pharmaceutically acceptable aqueous diluent to prepare the injectable formulation. As mentioned above, the pharmaceutically acceptable aqueous diluent may be selected independently from the group consisting of WFI (water for injection), D5W (5% dextrose in water), and normal saline, and Lactated Ringer's solution, among other things. The pharmaceutically acceptable aqueous diluent is chosen both for its ability to be tolerated by patients when delivered parenterally to patients, and for its suitability as a diluent for the pharmaceutical composition of the present invention.

Additionally, the injectable formulation of the present invention can also include antioxidants (for example, ascorbic acid, BHA (butylated hydroxyanisole) and preservatives (i.e., BHT (butylated hydroxytoluene)), vitamin E, TPGS, and the like) for enhancing chemical stability of the therapeutic compounds of Formula I.

Importantly, the compositions and formulations of this invention provide improved bioavailability for the therapeutic compounds of Formula I, as compared with unformulated compounds. In part this is due to the fact that, upon introduction into an aqueous solution, the surfactant within pharmaceutical composition forms self-association structures, including micelles, in which the therapeutic compounds of Formula I remain solubilized, at least for a period long enough to allow for administration of the injectable formulation to a patient in need of such treatment. Hence, in one preferred embodiment the pharmaceutical compositions of the instant invention are prepared so that they can (a) solubilize the therapeutic compounds of Formula I, (b) form micelles when introduced into aqueous solutions, and (c) maintain the therapeutic compounds of Formula I in solution in the injectable formulations, when these compositions are combined with pharmaceutically acceptable aqueous diluents.

Thus, in yet another aspect of the instant invention, methods are provided for preparing an injectable formulation for parenteral administration to patients in need of such treatment. In one embodiment, the method comprises the following steps: (a) solubilizing or dispersing an effective amount of one or more hydrophobic therapeutic agents of Formula I in a solubilizer to form a stable solution or dispersion, and mixing the resulting solution or dispersion with a sufficient amount of a pharmaceutically acceptable diluent to form a metastable solution. In preferred embodiments, the solubilizer is a non-ionic surfactant optionally in admixture with a viscosity reducing agent.

Importantly, the methods of preparing compositions and formulations of the invention can be scaled to any volume desired. Thus, even if a method specifies that the total volume of the solution is 100 mL, the composition or formulation can be prepared as a 1 mL sample by proportionally decreasing each component of the formulation by a factor of 100. For example, if 10 grams of a compound of Formulas I is to be dissolved in a 100 mL volume of the solution comprising a non-ionic surfactant and, optionally, a viscosity reducing agent, to prepare the concentrated pharmaceutical composition, then 0.1 grams of the compound could be dissolved in a 1 mL sample of that same solution. Similarly, if 10 mL of the concentrated pharmaceutical composition is normally added to 100 ml of D5W to prepare the injectable formulation, then a sample of the injectable formulation can be made by adding 0.1 mL of the pharmaceutical composition to 1 ml of D5W.

Preferably the mixing of the two solutions is accomplished by simple inversion and/or agitation of the combined solutions in a sealed container until a uniform solution is obtained. Ideally the mixing process is done by hand. If, however, more aggressive mixing is required to obtain a uniform solution, the combined liquids can be mixed by any suitable mechanical means, including mechanized stirring, shaking or vortexing, or through the use of ultrasound or other vibrations.

Advantageously, the injectable formulations of the instant invention can be prepared by combining the primary formulation with pharmaceutically acceptable aqueous diluents that are already in containers suitable for administration by intravenous injection. In particular, the pharmaceutical compositions can be introduced directly into a suitable, pharmaceutically acceptable diluent contained within an intravenous injection bag (i.v. bag), whereupon the two solutions are mixed to form a uniform injectable formulation. Preferably, the i.v. bag containing the diluent solution is composed of a substantially inert material, or is lined with a substantially inert material, such as a polyolefin. Ideally, chemical components in the i.v. bag should not leach from the bag itself into the injectable formulation contained within it and thereby contaminate the injectable formulation. Such leaching can be a problem with i.v. bags made with polyvinyl chloride (PVC) as the solution contacting surface. Additionally, the i.v. bag used should be resistant to attack by the non-ionic surfactant, and, if present, the viscosity reducing agent, used to solubilize the compounds of Formula I. Furthermore, the internal walls of the i.v. bag used should not possess substantial affinity for the compounds of Formula I, so that the concentration of the therapeutic agents remains constant in the injectable formulation contained within the i.v. bag.

Examples of suitable i.v. bags that can be used to prepare the injectable formulations of the instant invention include the polyolefin-lined i.v. bags manufactured by B. Braun Medical, Inc., of Bethlehem, Pa., U.S.A. These i.v. bags are known by the trademark PAB®, and are available with either D5W or 0.9% saline (normal saline) in pre-measured quantities, that only partially fill the i.v. bag, leaving room for the addition of the concentrated pharmaceutical compositions of the instant invention.

### Kits for Preparing Injectable Formulations

In still another aspect of the present invention, kits specifically designed for the preparation of the injectable formulations of the invention are provided. The kit of the present invention comprises, in a compartmentalized container, a compound of Formula I, a semi-solid or liquid solubilizer, and optionally a viscosity reducing agent, and also optionally instructions for using the kit for the preparation of an injectable formulation of the present invention suitable for intravenous injection into a mammal. In the kit of the present invention, the various components can be in the same or different compartments. For example, in one embodiment, the kit of the present invention comprises a single vial or bottle in a compartmentalized container, and instructions for its use. In this embodiment, the vial or bottle contains a measured volume of sterile “drug product,” or a concentrated pharmaceutical composition or primary formulation, of the instant invention, as described above, which comprises in solution or dispersion an effective amount of a therapeutic compound of Formula I in a solubilizer (e.g., a surfactant, preferably non-ionic surfactant) or in a liquid vehicle that comprises a non-ionic surfactant and optionally, a viscosity reducing agent. The instructions provide a detailed protocol or procedure on how to use the drug product (i.e., concentrated pharmaceutical composition) to prepare an injectable formulation of the therapeutic compounds of Formula I. The instructions also optionally provide detailed protocols or procedures on how to administer the injectable formulation so prepared.

In another embodiment of the kit of the present invention, an effective amount of a therapeutic compound of Formula I is in a different compartment from the semi-solid or liquid solubilizer (e.g., a surfactant, preferably non-ionic surfactant), and optionally from the viscosity reducing agent. Thus, for example, the kit can include two vials or bottles situated in a compartmentalized container, and instructions for their use. In this embodiment, one vial or bottle contains a dry powdered therapeutic compound of Formula I, and the other vial or bottle contains a measured volume of a liquid vehicle comprising a non-ionic surfactant and, optionally, a viscosity reducing agent. The instructions provide a detailed protocol or procedure on how to use the liquid vehicle to solubilize the powdered compound and thereby prepare “reconstituted drug product.” The instructions also provide a detailed protocol or procedure on how to use this reconstituted drug product (i.e., a pharmaceutical composition) to prepare an injectable formulation of the therapeutic compounds of Formula I. The instructions also optionally provide detailed protocols or procedures on how to administer the injectable formulation so prepared.

The kits of the present invention may optionally contain additional items, such as sterile filtration devices, and polyolefin lined, i.v. bags containing a pharmaceutically acceptable diluent, such as D5W or normal saline, which are intended to be used in preparing an injectable formulation of the therapeutic compounds of Formula I.

### Methods of Treatment

In yet another aspect of the present invention, the injectable formulations described are used in treating or delaying the onset of symptoms of an abnormal condition in a patient in need of such treatment. In certain embodiments of the present invention the patient is a mammal. In specific embodiments of the present invention the patient is a human. In most embodiments of the present invention, the pharmaceutical composition as described above is used to prepare an injectable formulation, also as described above, and the injectable formulation is used to treat or delay the onset of symptoms of an the abnormal condition in the patient. In all embodiments, the injectable formulation is administered parenterally, and generally intravenously, to the patient in need of such treatment. Abnormal conditions that may be treated with these formulations include cell proliferative disorders and diseases and hyperproliferative diseases, such as cancers and neoplasias.

As used herein, the term “treating” refers to the method of the invention having a therapeutic effect and at least partially alleviating or abrogating the abnormal condition in an organism, or delaying the onset of symptoms of this abnormal condition.

The term “therapeutic effect,” as used herein, refers to the inhibition of cell growth or proliferation, causing or contributing to an abnormal condition. The term “therapeutic effect” also refers to the inhibition of factors causing or contributing to the abnormal condition. A therapeutic effect relieves to some extent one or more of the symptoms of the abnormal condition.

As used herein, the term “mammal” as used herein preferably refers to such organisms as mice, rats, rabbits, guinea pigs, goats, sheep, horses, and cows, for example; more preferably to dogs, cats, monkeys, and apes; and most preferably to humans.

The term “cell proliferative disorder or disease,” or, alternatively “hyperproliferative disorder or disease,” as used herein, refers to a disorder or disease where an excess cell proliferation of one or more subset of cells in a multicellular organism occurs, resulting in harm (e.g., discomfort or decreased life expectancy) to the multicellular organism. The excess cell proliferation can be determined by reference to the general population and/or by reference to a particular patient (e.g., at an earlier point in the patient's life). Hyperproliferative disorders can occur in different types of animals and in humans, and produce different physical manifestations depending upon the affected cells. Hyperproliferative disorders include cancers, blood vessel proliferative disorders, fibrotic disorders, and autoimmune disorders.

In reference to the treatment of abnormal cell proliferative conditions, a therapeutic effect refers to one or more of the following: (a) a reduction in tumor size; (b) inhibition (i.e., slowing or stopping) of tumor metastasis; (c) inhibition of tumor growth; and (d) relieving to some extent one or more of the symptoms associated with the abnormal condition. Compounds demonstrating efficacy against leukemias can be identified as described herein, except that rather than inhibiting metastasis, the compounds may instead slow or decrease cell proliferation or cell growth.

As used herein, the term “abnormal condition” refers to a function in the cells or tissues of a patient that deviates from their normal functions in that patient. An abnormal condition can relate to cell proliferation as described herein.

The pharmaceutical compositions and formulations of the hydrophobic therapeutic agents of Formula I can be used as antimetastatic or anticancer agents. The pharmaceutical compositions and formulations can also be used in the treatment of other hyperproliferative diseases and disorders.

The proper dosage of therapeutic compounds to be used in the treatment of diseases and disorders depends on various factors such as the type of disease being treated, the particular formulation being used, the route by which the therapeutic compound is being delivered, and the mass and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can then be used to more accurately determine useful doses in humans.

For the treatment of cancers and other neoplastic diseases and disorders the expected daily dose of a therapeutic compound of Formula I is between 0.05 to 500 mg/kg per day, preferably 0.05 to 100 mg/kg per day. The therapeutic compound of Formula I can be delivered less frequently provided plasma levels of the active moiety are sufficient to maintain therapeutic effectiveness.

The injectable formulations of the instant invention are to be administered parenterally to patients in need of such treatment. The exact route of administration will be determined by the nature of the abnormal condition exhibited by the patient, but in one set of embodiments, administration is by way of intravenous injection.

Since hypersensitivity reactions may result from administration of polyoxyl 35 castor oil, optionally, the patient is premedicated with a medicament that reduces or eliminates hypersensitivity reactions. Thus, the present invention provides a method of treating a patient comprising: premedicating the patient with a medicament that reduces or eliminates hypersensitivity reactions, and administering to the patient an effective amount of an injectable formulation of the present invention. For this purpose, standard medical protocols, such as those developed for the administration of paclitaxel (TAXOL) to cancer patients, can be used with minor adjustments as will be apparent to a skilled artisan. For example, the premedicating step can comprise (1) orally administering an effective amount of dexamethasone approximately 12 and 6 hours prior to parenterally administering the injectable formulation of the present invention; and (2) after administering the dexamethasone, intravenously administering (i) an effective amount of an antihistamine and (ii) an effective amount of cimetidine or ranitidine, prior to parentally administering the injectable formulation comprising a therapeutic compound of Formula I.

The amounts of injectable formulations to be administered will be adjusted in order to deliver a therapeutically acceptable amount of a therapeutic compound of Formula I, i.e., in an amount sufficient to promote apoptosis and/or to reduce the proliferation of abnormal cells.

Generally, the toxicity profile and therapeutic efficacy of the therapeutic agents can be determined by standard pharmaceutical procedures in suitable cell models or animal models. As is known in the art, the LD₅₀ represents the dose lethal to about 50% of a tested population. The ED₅o is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but remains significantly below the LD₅₀ dosage level, as determined from cell or animal models.

Typically, the therapeutic compounds of Formula I delivered via the injectable formulations of the present invention can be effective at an amount of from about 0.05 mg to about 4000 mg per day, preferably from about 0.1 mg to about 2000 mg per day. However, the amount can vary with the body weight of the patient treated and the state of disease conditions. The injectable formulations may be administered at once, or may be divided into a number of smaller doses to be administered at predetermined intervals of time.

The pharmaceutical compositions and formulations of the present invention comprising at least one therapeutic compound of Formula I can also desirably be administered in combination with other therapeutic treatments including conventional surgery to remove a tumor, radiation and/or chemotherapy treatments wherein a compound or composition of the present invention can be administered to extend the dormancy of micrometastases and to stabilize and inhibit the growth of any residual primary tumor.

In the case of combination therapy, a therapeutically effective amount of another therapeutic compound can be administered in a separate pharmaceutical composition, or alternatively included in the pharmaceutical composition according to the present invention. The pharmacology and toxicology of other therapeutic compositions are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

### EXAMPLES

The examples below are not limiting and are merely representative of various aspects and features of the present invention. The examples demonstrate methods of testing the solubility of the hydrophobic pharmaceutical agents in the formulations. In addition, the examples illustrate preparation procedures for the formulations of the invention.

### Example 1

### Solubility of 5-Chloro-N-[2-(4-chloro-naphtalen-1-yloxy)-5-trifluoromethyl-phenyl]-2-hydroxy-benzamide in various solvents

Pure, dry powdered 5-Chloro-N-[2-(4-chloro-naphtalen-1-yloxy)-5-trifluoromethyl-phenyl]-2-hydroxy-benzamide was weighed out and added to solvent solution. The solutions were mixed at room temperature (about 25° C.). The amount of 5-Chloro-N-[2-(4-chloro-naphtalen-1-yloxy)-5-trifluoromethyl-phenyl]-2-hydroxy-benzamide dissolved in the solvent solutions was determined.

The amounts of 5-Chloro-N-[2-(4-chloro-naphtalen-1-yloxy)-5-trifluoromethyl-phenyl]-2-hydroxy-benzamide dissolved are given in the Table below.

TABLE 1
Solubility of 5-Chloro-N-[2-(4-chloro-naphtalen-1-yloxy)-5-
trifluoromethyl-phenyl]-2-hydroxy-benzamide in selected solvents

| | Maximum Solubility | |
|---|---|---|
| Solvent | at 25° C. (mg/mL) | Comments |
| Water | Insoluble | |
| Methanol | 5.34 | precipitate forms upon contact |
| | | with water |
| Ethyl Acetate | 194.68 | |
| Hexane | 0.38 | |
| DMF | 341.26 | precipitate forms upon contact |
| | | with water |
| Toluene | 102.00 | |
| Propylene glycol | <5 | Unstable; dark color; |
| | | precipitate forms upon contact |
| | | with water |
| Glycerol formal | <5 | Unstable; dark color; |
| | | precipitate forms upon contact |
| | | with water |
| VP-16 | >50 | precipitate forms upon contact |
| | | with water |
| NMP | >50 | precipitate forms upon contact |
| | | with water |
| Ethyl benzoate | >5 | precipitate forms upon contact |
| | | with water |
| Benzyl benzoate | >5 | precipitate forms upon contact |
| | | with water |
| Benzyl alcohol | >5 | precipitate forms upon contact |
| | | with water |
| Sesame Oil | Insoluble | |

### Example 2

### Phase solubility of 5-Chloro-N-[2-(4-chloro-naphtalen-1-yloxy)-5-trifluoromethyl-phenyl]-2-hydroxy-benzamide, solubilized in various surfactants or with a complexing agent, upon addition to D5W

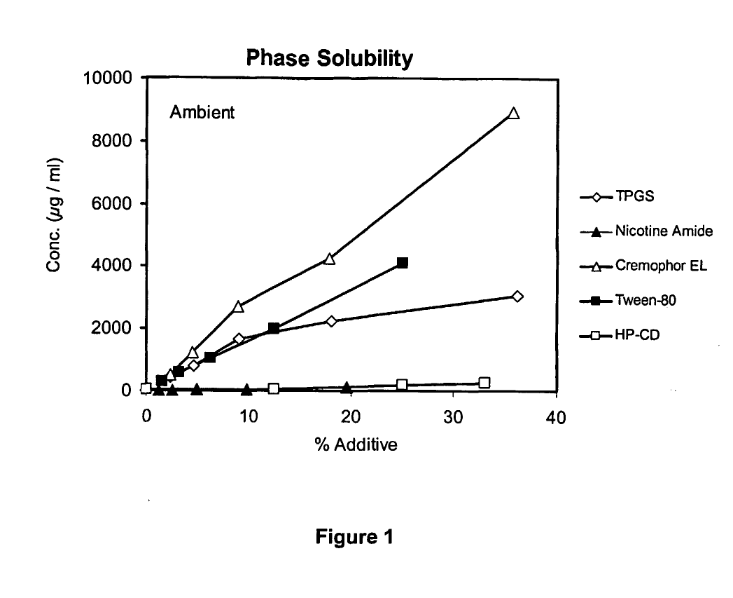

Pure, powdered 5-Chloro-N-[2-(4-chloro-naphtalen-1-yloxy)-5-trifluoromethyl-phenyl]-2-hydroxy-benzamide was weighed out and mixed at room temperature with (1) Cremophor EL:Ethanol (50:50), (2) Nicotinamide, (3) TPGS, or (4) Tween-80. Aliquots of solubilized compound were combined with D5W in increasing amounts. The concentration of dissolved 5-Chloro-N-[2-(4-chloro-naphtalen-1-yloxy)-5-trifluoromethyl-phenyl]-2-hydroxy-benzamide in the final formulations was determined and the results are shown in FIG. 1.

### Example 3

### Proposed Preparation of Pharmaceutical Compositions

A protocol for an exemplary method used to prepare pharmaceutical compositions of the invention is given below.

### Generic Preparation of Pharmaceutical Compositions:

1. Weigh appropriate amounts of the compound of Formula I to be solubilized.

2. Add an appropriate amount of a 1:1 mixture of Cremophor EL and ethanol to dissolve the drug.

3. Mix thoroughly and filter through a 0.2 μm polyvinylidene fluoride (PVDF) sterile filter unit (such as a Millipore Durapore filter, Billerica, Mass., USA).

4. Aliquot appropriate volumes into sterile vials.

5. Seal vials aseptically.

6. Store filled vials in the dark at temperatures of 25° C. or below.

### Example 4

### Stability of 5-Chloro-N-[2-(4-chloro-naphtalen-1-yloxy)-5-trifluoromethyl-phenyl]-2-hydroxy-benzamide solubilized in Cremophor EL:EtOH

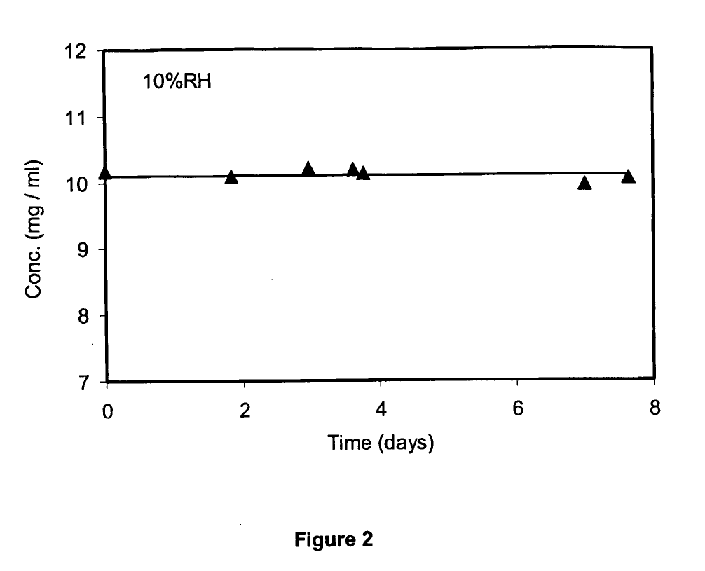

A solution of 10 mg 5-Chloro-N-[2-(4-chloro-naphtalen-1-yloxy)-5-trifluoromethyl-phenyl]-2-hydroxy-benzamide per mL of Cremophor:EtOH (1:1) was prepared by mixing 330.3 mg of 5-Chloro-N-[2-(4-chloro-naphtalen-1-yloxy)-5-trifluoromethyl-phenyl]-2-hydroxy-benzamide with 15.03 grams of Cremophor EL and 15.03 grams of 95% Ethanol (USP Grade). The resulting mixture was incubated at 60° C. for 8 days. Periodic samples were taken and the amount of 5-Chloro-N-[2-(4-chloro-naphtalen-1-yloxy)-5-trifluoromethyl-phenyl]-2-hydroxy-benzamide was determined. The results, shown in FIG. 2, indicate that the concentration of 5-Chloro-N-[2-(4-chloro-naphtalen-1-yloxy)-5-trifluoromethyl-phenyl]-2-hydroxy-benzamide was stable over the entire period. Stability of this duration at 60° C. predicts a prolonged stability of room temperature (i.e., 25° C.), likely more than one year.

### Example 5

### Preparation of Injectable Formulations

The protocol for the generic method used to prepare injectable formulations of the invention for parenteral administration is given below.

### Generic Injectable Formulation Preparation Procedure:

1. Remove a specific volume of dissolved drug (pharmaceutical composition) from a sealed vial using a sterile syringe and aseptic technique.

2. Add the dissolved drug to a polyolefin lined i.v. bag containing 500 ml of D5W.

3. Mix well for 3 minutes.

4. Administer the injectable formulation, preferably immediately after mixing.

5. Store formulations in the dark at room temperature until administered.

6. If the formulation is not administered within 12 hours, discard.

### Example 6

### Stability of an injectable formulation comprising 5-Chloro-N-[2-(4-chloro-naphtalen-1-yloxy)-5-trifluoromethyl-phenyl] -2-hydroxy-benzamide solubilized in Cremophor EL:EtOH, and diluted in D5W

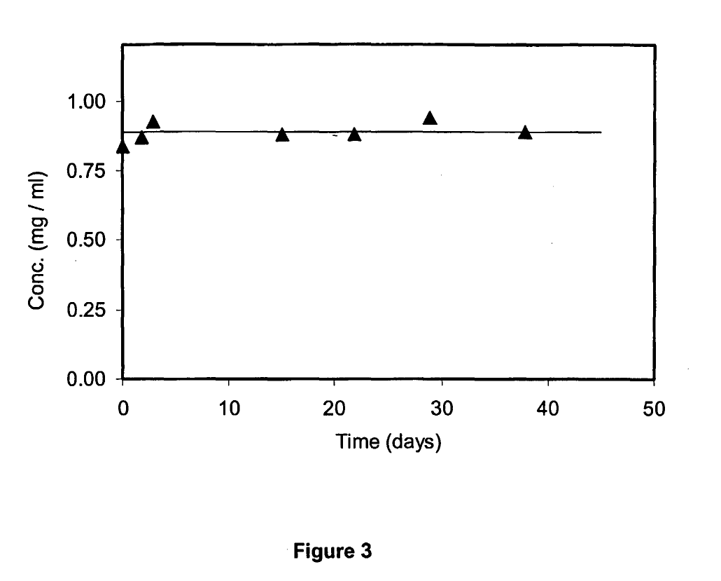

A solution of 10 mg 5-Chloro-N-[2-(4-chloro-naphtalen-1-yloxy)-5-trifluoromethyl-phenyl]-2-hydroxy-benzamide per mL of Cremophor:EtOH (1:1) was prepared by mixing 330.3 mg of 5-Chloro-N-[2-(4-chloro-naphtalen-1-yloxy)-5-trifluoromethyl-phenyl]-2-hydroxy-benzamide with 15.03 grams of Cremophor EL and 15.03 grams of 95% Ethanol (USP Grade). 10 ml of this solution was diluted 1:9 by the addition of 90 ml of D5W, and the resulting formulation was mixed by inversion until the solution appeared uniformly clear. The resulting mixture—an injectable formulation—was incubated at room temperature (approximately 25° C.) for 38 days. Periodic samples were taken and the amount of 5-Chloro-N-[2-(4-chloro-naphtalen-1-yloxy)-5-trifluoromethyl-phenyl]-2-hydroxy-benzamide was determined. The results, shown in FIG. 3, indicate that the concentration of 5-Chloro-N-[2-(4-chloro-naphtalen-1-yloxy)-5-trifluoromethyl-phenyl]-2-hydroxy-benzamide in the injectable formulation was stable over the entire period.

### Example 7

### Examples of Injectable Formations

| | Injectable | | Injectable | | Injectable | |
|---|---|---|---|---|---|---|
| | Formulation | | Formulation | | Formulation | |
| Component | A | | B | | C | |
| D5W | 250 | mL | 500 | mL | 500 | mL |
| Primary Formulation* | 56 | mL | 56 | mL | 28 | mL |
| (10 mg drug per mL of | | | | | | |
| 1:1 Cremophor:EtOH) | | | | | | |
| Concentration of | 8.45% | | 4.65% | | 2.45% | |
| Cremophor | | | | | | |
| Concentration of | 8.45% | | 4.65% | | 2.45% | |
| EtOH | | | | | | |
| Concentration of | 1.83 | mg/mL | 1.01 | mg/mL | 0.530 | mg/mL |
| drug | | | | | | |

*Primary formulation = 10 mg 5-Chloro-N-[2-(4-chloro-naphtalen-1-yloxy)-5-trifluoromethyl-phenyl]-2-hydroxy-benzamide per mL of 1:1 Cremophor EL:EtOH

One skilled in the art would readily appreciate that the present invention is well adapted to carry out the objectives of the invention, and obtain the ends and advantages mentioned, as well as those inherent therein. The methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

In various parts of this disclosure, certain publications or patents are discussed or cited. The mere discussion of, or reference to, such publications or patents is not intended as admission that they are prior art to the present invention.

What is claimed is:

1. A pharmaceutical composition comprising a solution or a dispersion of a therapeutically effective amount of a compound of Formula I 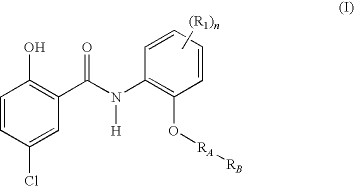
in a semi-solid or liquid solubilizer, wherein
R_{A} is a bond, or a straight chain or branched C₁₋₆ alkyl;
R_{B} is, phenyl or napthyl, optionally substituted with 1-7 halo or C₁₋₆ haloalkyl substituents; and
R₁ is, independently, halo or C₁₋₆ haloalkyl, and n=0-4.

2. The pharmaceutical composition of claim 1, further comprising in admixture an aqueous diluent.

3. The pharmaceutical composition of claim 1, wherein said solubilizer is a non-ionic surfactant.

4. The pharmaceutical composition of claim 1, wherein the pharmaceutically acceptable nonionic surfactant is selected from the group consisting of:
a polyethoxylated castor oil,
a polysorbate (Tween),
a sorbitan ester (Span),
a polyoxyethylene hydroxystearate
a polyoxyethylene stearate (Myrj), and
a polyoxyethylene fatty acid ether (Brij).

5. The pharmaceutical composition of claim 1, further comprising one or more pharmaceutically acceptable viscosity reducing agents in a sufficient quantity to render the composition readily syringable or filterable.

6. The pharmaceutical composition of claim 5, in which the one or more pharmaceutically acceptable viscosity reducing agents are selected from the group consisting of:
C₁₋₅ alkanols,
monoesters of glycerol, and
aliphatic mono carboxylic acids.

7. The pharmaceutical composition of claim 6, wherein the pharmaceutically acceptable viscosity reducing agent is ethanol.

8. The pharmaceutical composition of claim 6, wherein the ratio of nonionic surfactant to viscosity reducing agent is about 10:1 to about 1:10 (v/v).

9. The pharmaceutical composition of claim 1, further comprising a pharmaceutically acceptable excipient to provide a pH of about 4 to about 9.

10. An injectable formulation comprising the pharmaceutical composition of claim 1 diluted in a pharmaceutically acceptable aqueous diluent.

11. The injectable formulation of claim 10, wherein the ratio of the pharmaceutical composition to the pharmaceutically acceptable aqueous diluent is at least about 1:10 (v/v).

12. The injectable formulation of claim 11, wherein said ratio of the pharmaceutical composition to the pharmaceutically acceptable aqueous diluent is about 1:10 to about 1:500 (v/v).

13. The injectable formulation of claim 10 wherein said pharmaceutically acceptable aqueous diluent is selected from the group consisting of water for injection (WFI), sterile water for injection (SWFI), 5% dextrose in water (D5W), normal saline, and 5% dextrose in ½ normal saline (D5W½N).

14. The pharmaceutical composition of claim 1, wherein
R_{A} is a straight chain or branched C₁₋₆ alkyl; and
R_{B} is phenyl, optionally substituted with 1-5 halo or C₁₋₆ haloalkyl substituents.

15. The pharmaceutical composition of claim 1, wherein
R_{A} is a bond; and
R_{B} is napthyl, optionally substituted with 1-7 halo or C₁₋₆ haloalkyl substituents.

16. The pharmaceutical composition of claim 1, wherein the compound of Formula I is selected from the group consisting of:

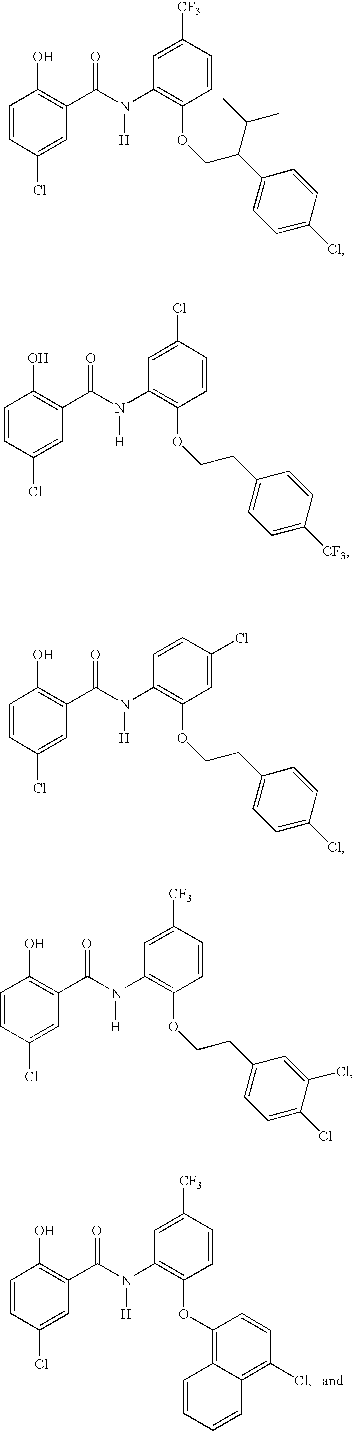

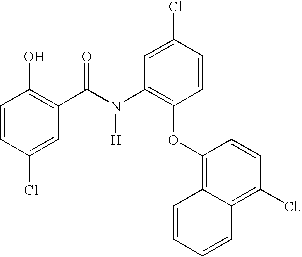

17. The pharmaceutical composition of claim 1, wherein the compound of Formula I is selected from the group consisting of
5-Chloro-N-{2-[2-(4-chloro-phenyl)-3-methyl-butoxy]-5-trifluoromethyl-phenyl}-2-hydroxy-benzamide,
5-Chloro-N-{5-chloro-2-[2-(4-trifluoromethyl-phenyl)-ethoxy]-phenyl}-2-hydroxy-benzamide,
5-Chloro-N-{4-chloro-2-[2-(4-chloro-phenyl)-ethoxy]-phenyl}-2-hydroxy-benzamide,
5-Chloro-N-{2-[2-(3,4-dichloro-phenyl)-ethoxy]-5-trifluoromethyl-phenyl}-2-hydroxy-benzamide
5-Chloro-N-[2-(4-chloro-naphtalen-1-yloxy)-5-trifluoromethyl-phenyl]-2-hydroxy-benzamide, and
5-Chloro-N-[5-chloro-2-(4-chloro-naphthalen-1-yloxy)-phenyl]-2-hydroxy-benzamide.

18. A pharmaceutical formulation of a compound of Formula I, 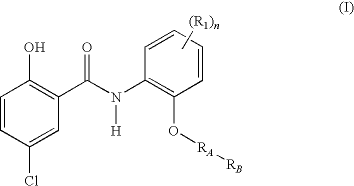
in which said compound is dissolved in a pharmaceutically acceptable liquid vehicle comprising a nonionic surfactant and a viscosity reducing agent in a quantity sufficient to dissolve the compound and form micelles when the composition is mixed with a pharmaceutically acceptable aqueous diluent, wherein
R_{A} is a bond, or a straight chain or branched C₁₋₆ alkyl;
R_{B} is, phenyl or napthyl, optionally substituted with 1-7 halo or C₁₋₆ haloalkyl substituents; and
R₁ is, independently, halo or C₁₋₆ haloalkyl, and n=0-4.

19. The pharmaceutical composition of claim 18 wherein said nonionic surfactant is a polyethoxylated castor oil and said viscosity reducing agent is ethanol.

20. The pharmaceutical composition of claim 19 which, when added to a pharmaceutically acceptable aqueous diluent, forms a stable or metastable micellar solution suitable for parenteral administration to patients in need of such treatment.

21. An injectable formulation comprising the pharmaceutical formulation of claim 19 diluted in a pharmaceutically acceptable aqueous diluent.

22. A pharmaceutical composition comprising a therapeutically effective amount of a compound of Formula II 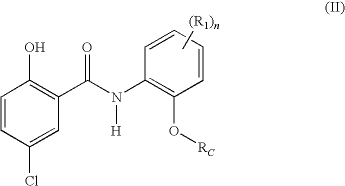
and a pharmaceutically acceptable nonionic surfactant in a quantity sufficient to dissolve the compound and form micelles when the composition is mixed with a pharmaceutically acceptable aqueous diluent, wherein 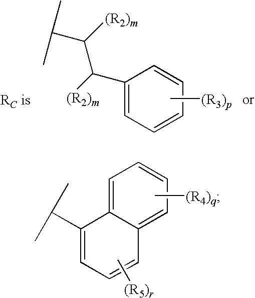
R₁ is, independently, halo or C₁₋₆ haloalkyl, and n=0-4;
R₂ is, independently, straight chained or branched C₁₋₄ alkyl, and m=0-2;
R₃ is, independently, halo or C₁₋₆ haloalkyl, and p=0-5;
R₄ is, independently, halo or C₁₋₆ haloalkyl, and n=0-4; and
R₅ is, independently, halo or C₁₋₆ haloalkyl, and n=0-3.

23. The pharmaceutical composition of claim 22 further comprising a viscosity reducing agent.

24. The pharmaceutical composition of claim 23 wherein said nonionic surfactant is polyoxyl 35 castor oil and said viscosity reducing agent is ethanol.

25. A kit comprising, in a compartmentalized carrier,
a container containing a compound of Formula I in a liquid vehicle, said liquid vehicle comprising a nonionic surfactant and a viscosity reducing agent; 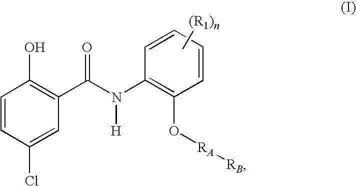
and instructions for the use of the kit;
wherein
R_{A} is a bond, or a straight chain or branched C₁₋₆ alkyl;
R_{B} is, phenyl or napthyl, optionally substituted with 1-7 halo or C₁₋₆ haloalkyl substituents; and
R₁ is, independently, halo or C₁₋₆ haloalkyl, and n=0-4.

26. A kit comprising, in a compartmentalized carrier,
a compound of Formula I in a first container 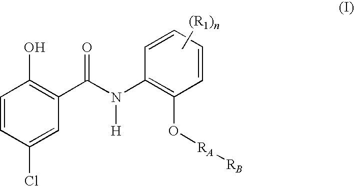
a liquid vehicle comprising a nonionic surfactant and a viscosity reducing. agent in a second container, and
said first and second containers being positioned within the compartmentalized container, and
said kit optionally containing instructions for its use;
wherein
R_{A} is a bond, or a straight chain or branched C₁₋₆ alkyl;
R_{B} is, phenyl or napthyl, optionally substituted with 1-7 halo or C₁₋₆ haloalkyl substituents; and
R₁ is, independently, halo or C₁₋₆ haloalkyl, and n=0-4.

27. The kit of claim 26, wherein said nonionic surfactant is polyethoxylated castor oil, and said viscosity reducing agent is an alcohol.

28. The kit of claim 27, wherein said polyethoxylated castor oil is polyoxyl 35 castor oil, and said alcohol is ethanol.

29. The kit of claim 26, wherein the ratio of said nonionic surfactant to said viscosity reducing agent, in said second container, is from about 1:10 to about 10:1.

30. The kit of claim 26, further comprising a pharmaceutically acceptable excipient in said second container to provide a pH of about 4 to about 9.

31. The kit of claim 26, in which said first and second containers are sealed with tops configured in such a manner that liquids can be conveniently introduced into, or removed from, the containers by way of syringes.

32. The kit of claim 31, in which said tops can be punctured by a syringe needle.

33. A method of preparing a pharmaceutically acceptable injectable formulation of a compound of Formula I, comprising the steps of:
a. dissolving a compound of Formula I in a pharmaceutically acceptable vehicle comprising a nonionic surfactant and a viscosity reducing agent, to form a reconstituted drug mixture, and
b. diluting said reconstituted drug mixture in a pharmaceutically acceptable aqueous diluent.

34. The method of claim 33, wherein said aqueous solution is selected from the group consisting of water for injection (WFI), sterile water for injection (SWFI), 5% dextrose in water (D5W), or normal saline.

35. The method of claim 34, wherein a specific volume of said reconstituted drug mixture is diluted into said aqueous solution to create a pharmaceutically acceptable injectable formulation containing a specific amount (dose) of a compound of Formula I.

36. The method of claim 35, wherein the volume of said reconstituted drug mixture used to make said injectable formulation, is proportional to the mass of the patient in need of treatment that is to receive said injectable formulation.

37. A method of treating cancer comprising:
a. dissolving a compound of Formula I in a pharmaceutically acceptable vehicle comprising a nonionic surfactant and a viscosity reducing agent, to form a reconstituted drug mixture,
b. diluting said reconstituted drug mixture in a pharmaceutically acceptable aqueous diluent to create an injectable formulation, and
c. administering said injectable formulation parenterally to said patient in need of treatment.

38. The method of claim 37, wherein said pharmaceutically acceptable aqueous diluent is selected from the group consisting of water for injection (WFI), sterile water for injection (SWFI), 5% dextrose in water (D5W), or normal saline.

39. The method of claim 37, wherein said administering step is by intravenous injection.
